(19) United States
(12) Patent Application Publication
(10) Pub. No.: US 2023/0255587 A1
(43) Pub. Date: Aug. 17, 2023

(54) SYSTEM AND METHOD FOR AUTOMATICALLY MEASURING AND LABELING FOLLICLES DEPICTED IN IMAGE SLICES OF AN ULTRASOUND VOLUME

(71) Applicant: GE Precision Healthcare LLC, Wauwatosa, WI (US)

(72) Inventors: Fiona Schwab, Mattighofen (AT); Martin Mienkina, Neumarkt am Wallersee (AT)

(21) Appl. No.: 17/672,386

(22) Filed: Feb. 15, 2022

(51) Int. Cl. A61B 8/08; G06T 7/10; G06T 7/62; G06T 7/00; A61B 8/00; A61B 8/13

(52) U.S. Cl. CPC A61B 8/085; G06T 7/10; G06T 7/62; G06T 7/0012; A61B 8/463; A61B 8/13; A61B 8/4461; G06T 2207/10132; G06T 2207/20092

(57) ABSTRACT

Systems and methods for automatically measuring and labeling follicles depicted in two-dimensional (2D) image slices of an ultrasound volume are provided. The method includes receiving an ultrasound volume of an ovary having follicles. The method includes causing a display to present, one at a time, 2D image slices extracted from the ultrasound volume. The method includes receiving a user indication identifying a follicle at a maximum size in a first 2D image slice of the ultrasound volume presented at the display. The method includes automatically segmenting the follicle in the ultrasound volume. The method includes automatically measuring the follicle in the first 2D image slice to generate a measurement presented at the display. The method includes automatically labeling the follicle in the first 2D image slice presented at the display and in any other of the parallel 2D image slices depicting the follicle that is subsequently presented at the display.

### FIELD

Certain embodiments relate to ultrasound imaging. More specifically, certain embodiments relate to a method and system for automatically measuring and labeling follicles depicted in two-dimensional (2D) image slices of an ultrasound volume.

### BACKGROUND

Ultrasound imaging is a medical imaging technique for imaging organs and soft tissues in a human body. Ultrasound imaging uses real time, non-invasive high frequency sound waves to produce a series of two-dimensional (2D) and/or three-dimensional (3D) images.

Women undergoing in vitro fertilization may receive injectable hormone medications to stimulate the development of eggs in the ovaries. The growth of follicles in the ovaries, which each contain a developing egg, may be monitored with ultrasound to prevent overstimulation of the ovaries. Typically, an ultrasound operator manually manipulates an ultrasound probe to acquire two-dimensional (2D) ultrasound images of an ovary. The ultrasound operator may manually perform a 2D follicle measurement (i.e., two perpendicular diameter measurements per follicle) of each follicle in the 2D ultrasound image that each follicle is at its maximum size. However, the workflow of identifying and measuring each follicle in an ovary may be time consuming and prone to error. For example, performing multiple measurements of a same follicle in different ultrasound images is a common problem using the typical workflow.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for automatically measuring and labeling follicles depicted in two-dimensional (2D) image slices of an ultrasound volume, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to automatically measure and label follicles depicted in two-dimensional (2D) image slices of an ultrasound volume, in accordance with various embodiments.

FIG. 2 is a block diagram of an exemplary medical workstation that is operable to automatically measure and label follicles depicted in two-dimensional (2D) image slices of an ultrasound volume, in accordance with various embodiments.

FIG. 3 is an exemplary display of a two-dimensional (2D) image slice of an ultrasound volume depicting a first follicle of an ovary, in accordance with various embodiments.

FIG. 4 is an exemplary display of a two-dimensional (2D) image slice of an ultrasound volume depicting a first follicle of an ovary being measured and labeled, in accordance with various embodiments.

FIG. 5 is an exemplary display of a two-dimensional (2D) image slice of an ultrasound volume depicting a first measured and labeled follicle and a second follicle of an ovary, in accordance with various embodiments.

FIG. 6 is an exemplary display of a two-dimensional (2D) image slice of an ultrasound volume depicting a second measured and labeled follicle and a third follicle of an ovary, in accordance with various embodiments.

FIG. 7 is an exemplary display of a two-dimensional (2D) image slice of an ultrasound volume depicting second and third measured and labeled follicles of an ovary, in accordance with various embodiments.

FIG. 8 is an exemplary display of a two-dimensional (2D) image slice of an ultrasound volume depicting a third measured and labeled follicle of an ovary, in accordance with various embodiments.

FIG. 9 is a flow chart illustrating exemplary steps that may be utilized for automatically measuring and labeling follicles depicted in two-dimensional (2D) image slices of an ultrasound volume, in accordance with various embodiments.

### DETAILED DESCRIPTION

Certain embodiments may be found in a method and system for automatically measuring and labeling follicles depicted in two-dimensional (2D) image slices of an ultrasound volume. Aspects of the present disclosure have the technical effect of automatically measuring segmented follicles depicted in 2D image slices of an ultrasound volume. Various embodiments have the technical effect of automatically labeling measured follicles depicted in a plurality of 2D image slices of an ultrasound volume to prevent double-counting of the follicles.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, Contrast Enhanced Ultrasound (CEUS), and/or sub-modes of B-mode and/or CF such as Harmonic Imaging, Shear Wave Elasticity Imaging (SWEI), Strain Elastography, TVI, PDI, B-flow, MVI, UGAP, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphic Processing Unit (GPU), DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

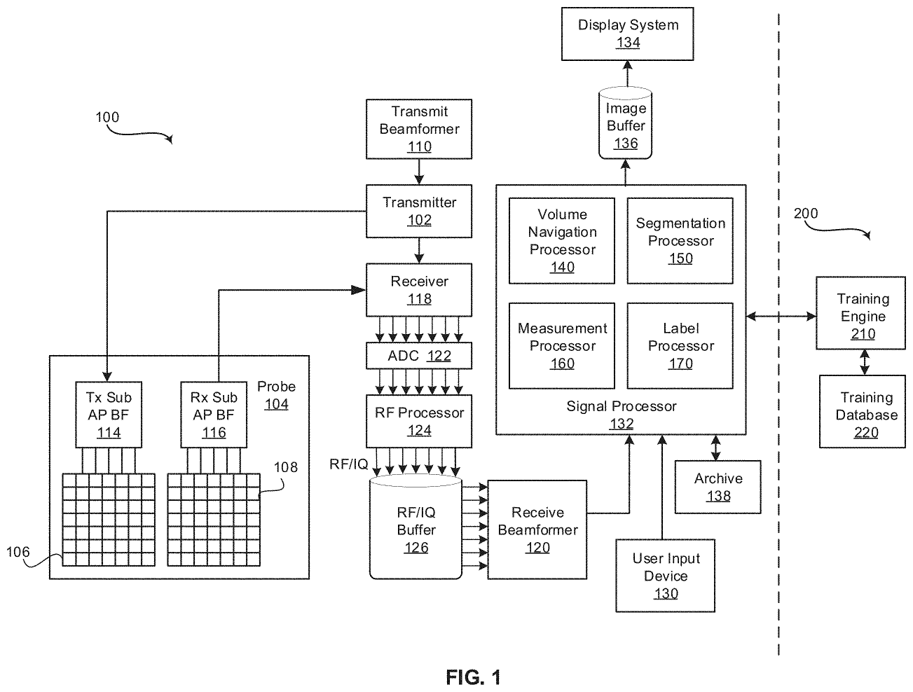

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to automatically measure and label follicles depicted in two-dimensional (2D) image slices of an ultrasound volume, in accordance with various embodiments. Referring to FIG. 1, there is shown an ultrasound system 100 and a training system 200. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may comprise a two dimensional (2D) array of piezoelectric elements. Additionally and/or alternatively, the ultrasound probe 104 may be a mechanically wobbling ultrasound probe 104, which may comprise a one dimensional (1D) array of piezoelectric elements mounted on a transducer assembly movable in a single plane. For example, the transducer assembly may be movable approximately 120 to 150 degrees by a motor driving gears, belts, and/or rope to pivot an axis or hub of the transducer assembly. In certain embodiments, the ultrasound probe 104 is a transvaginal mechanically wobbling ultrasound probe. The ultrasound probe 104 may comprise a group of transmit transducer elements 106 and a group of receive transducer elements 108, that normally constitute the same elements. The group of transmit transducer elements 106 may emit ultrasonic signals through oil and a probe cap and into a target. In a representative embodiment, the ultrasound probe 104 may be operable to acquire ultrasound image data covering at least a substantial portion of an anatomy, such as an ovary or any suitable anatomical structure. In an exemplary embodiment, the ultrasound probe 104 may be operated in a volume acquisition mode, where the transducer assembly of the ultrasound probe 104 is moved to acquire a plurality of parallel 2D ultrasound slices forming an ultrasound volume.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select protocols and/or templates, scroll through parallel 2D image slices of an ultrasound volume, pause and restart a cine loop of 2D image slices of an ultrasound volume, select follicles in 2D images slices of an ultrasound volume for measurement, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134 or the ultrasound probe 104, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise a volume navigation processor 140, a segmentation processor 150, a measurement processor 160, and a label processor 170. The signal processor 132 may be capable of receiving input information from a user input device 130 and/or archive 138, generating an output displayable by a display system 134, and manipulating the output in response to input information from a user input device 130, among other things. The signal processor 132, volume navigation processor 140, segmentation processor 150, measurement processor 160, and label processor 170 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a volume navigation processor 140 that comprises suitable logic, circuitry, interfaces and/or code that may be operable to extract and sequentially present, one at a time, 2D image slices from an ultrasound volume of an ovary or any suitable anatomical structure. For example, the volume navigation processor 140 may be configured to extract and separately present 2D image slices from the ultrasound volume at the display system 134 in response to user selections via the user input device 130 to navigate forward and/or backward through the ultrasound volume. As an example, an ultrasound operator may scroll through independently presented 2D image slices to locate follicles at a largest size for measurement and labeling. As another example, the volume navigation processor 140 may be configured to generate a cine loop of extracted 2D image slices for playback at the display system 134. The ultrasound operator may provide pause, play, rewind, and/or fast forward instructions via the user input device to the volume navigation processor 140 to control playback of the cine loop. The ultrasound operator may control playback of the cine loop to identify follicles at a largest size for measurement and labeling. The volume navigation processor 140 may be configured to store the extracted 2D image slices and/or cine loop at archive 138 and/or any suitable data storage medium.

The signal processor 132 may include a segmentation processor 150 that comprises suitable logic, circuitry, interfaces and/or code that may be operable to segment follicles in an ultrasound volume. The segmentation processor 150 may be configured to receive a user selection of a follicle depicted in a 2D image slice via the user input device 130. The segmentation processor 150 may be configured to segment the selected follicle in all 2D slices in which the selected follicle is depicted within the ultrasound volume. In this regard, the segmentation processor 150 may include, for example, artificial intelligence image analysis algorithms, one or more deep neural networks (e.g., a convolutional neural network such as u-net) and/or may utilize any suitable form of artificial intelligence image analysis techniques or machine learning processing functionality configured to provide segmentation of selected follicles or any suitable anatomical structure. Additionally and/or alternatively, the artificial intelligence image analysis techniques or machine learning processing functionality configured to segment the selected follicles in the ultrasound volume may be provided by a different processor or distributed across multiple processors at the ultrasound system 100 and/or a remote processor communicatively coupled to the ultrasound system 100. For example, the image segmentation functionality may be provided as a deep neural network that may be made up of, for example, an input layer, an output layer, and one or more hidden layers in between the input and output layers. Each of the layers may be made up of a plurality of processing nodes that may be referred to as neurons. For example, the image segmentation functionality may include an input layer having a neuron for each voxel of an ultrasound volume. The output layer may have a neuron corresponding to a follicle and/or any suitable anatomical structure. Each neuron of each layer may perform a processing function and pass the processed ultrasound image information to one of a plurality of neurons of a downstream layer for further processing. As an example, neurons of a first layer may learn to recognize edges of structure in the obtained ultrasound image. The neurons of a second layer may learn to recognize shapes based on the detected edges from the first layer. The neurons of a third layer may learn positions of the recognized shapes relative to landmarks in the obtained ultrasound image. The processing performed by the deep neural network may identify follicles and the location of the follicles in the obtained ultrasound image with a high degree of probability.

In an exemplary embodiment, the segmentation processor 150 may be configured to store the image segmentation information at archive 138 and/or any suitable storage medium. The segmentation processor 150 may be configured to provide the measurement processor 160 with the image segmentation information for measuring the selected follicle in the 2D image slice presented at the display system 134, as described below. The segmentation processor 150 may be configured to provide the label processor 170 with the image segmentation information for labeling the selected and measured follicle in all the 2D image slice presented at the display system 134 in which the selected and measured follicle is depicted, as described below.

The signal processor 132 may include a measurement processor 160 that comprises suitable logic, circuitry, interfaces and/or code that may be operable to measure the selected and segmented follicle in the 2D image slice presented at the display system 134. For example, the measurement processor 160 may be configured to receive the segmentation information from the segmentation processor 150 identifying the location and boundaries of the selected follicle in the 2D image slice. The measurement processor 160 may be configured to automatically place measurement calipers on the selected and segmented follicle in the 2D image slice presented at the display system to obtain perpendicular height and width diameter measurements. In various embodiments, the measurement processor 160 may further determine a mean of the height and width diameter measurements. As another example, the measurement processor 160 may be configured to receive the segmentation information from the segmentation processor 150 identifying the location and boundaries of the selected follicle in the ultrasound volume. The measurement processor 160 may be configured to determine a volume measurement of selected and segmented follicle in the ultrasound volume. The measurement processor 160 may be configured to cause the display system 134 to present the 2D follicle diameter measurements, volume measurement, and/or any suitable measurement. The measurement processor 160 may be configured to store the measurements at archive 138 and/or any suitable data storage medium.

The signal processor 132 may include a label processor 170 that comprises suitable logic, circuitry, interfaces and/or code that may be operable to label the selected, segmented, and measured follicle in the 2D image slice presented at the display system 134. For example, the label processor 170 may be configured to receive the segmentation information from the segmentation processor 150 identifying the location and boundaries of the selected follicle in the ultrasound volume. The label processor 170 may be configured to present a different label for each segmented follicle in each of the 2D image slices of the ultrasound volume presented at the display system 134 that includes the segmented follicle. The label may be textual, numerical, symbols, and/or any suitable label to distinguish between different selected, segmented, and measured follicles. The label processor 170 may be configured to superimpose the label(s) on or near the corresponding follicle(s) depicted in the 2D image slice presented at the display system 134. The label processor 170 may be configured to store the labeled 2D image slices and/or ultrasound volume at archive 138 and/or any suitable data storage medium.

The display system 134 may be any device capable of communicating visual information to a user. For example, a display system 134 may include a liquid crystal display, a light emitting diode display, and/or any suitable display or displays. The display system 134 can be operable to present the 2D image slices of the ultrasound volume extracted by the volume navigation processor 140, measurements provided by the measurement processor 160, labels provided by the label processor 170, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores ultrasound volumes, 2D image slices, instructions for extracting and separately presenting 2D image slices from an ultrasound volume, instructions for automatically segmenting selected follicle(s) in the ultrasound volume, instructions for measuring selected and segmented follicles in 2D image slice(s) and/or the ultrasound volume, and/or instructions for labeling selected, segmented, and measured follicles in 2D image slice(s), for example.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

Still referring to FIG. 1, the training system 200 may comprise a training engine 210 and a training database 220. The training engine 210 may comprise suitable logic, circuitry, interfaces and/or code that may be operable to train the neurons of the deep neural network(s) (e.g., artificial intelligence model(s)) inferenced (i.e., deployed) by the segmentation processor 150. For example, the artificial intelligence model inferenced by the segmentation processor 150 may be trained to automatically identify follicle(s) or other anatomical objections of interest depicted in an ultrasound volume using database(s) 220 of classified ultrasound volumes of follicles(s) or anatomical objects of interest.

In various embodiments, the databases 220 of training images may be a Picture Archiving and Communication System (PACS), or any suitable data storage medium. In certain embodiments, the training engine 210 and/or training image databases 220 may be remote system(s) communicatively coupled via a wired or wireless connection to the ultrasound system 100 as shown in FIG. 1. Additionally and/or alternatively, components or all of the training system 200 may be integrated with the ultrasound system 100 in various forms.

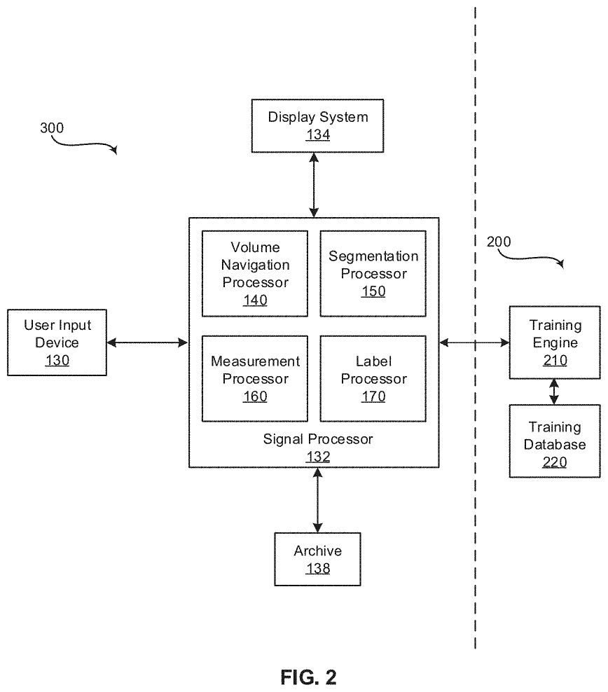

FIG. 2 is a block diagram of an exemplary medical workstation 300 that is operable to automatically measure and label follicles depicted in two-dimensional (2D) image slices of an ultrasound volume, in accordance with various embodiments. In a representative embodiment, components of the medical workstation 300 and training system 200 may share various characteristics with components of the ultrasound system 100 and training system 200, as illustrated in FIG. 1 and described above. Referring to FIG. 2, the medical workstation 300 comprises a display system 134, a signal processor 132, an archive 138, and a user input device 130, among other things. Components of the medical workstation 300 may be implemented in software, hardware, firmware, and/or the like. The various components of the medical workstation 300 may be communicatively linked. Components of the medical workstation 300 may be implemented separately and/or integrated in various forms. For example, the display system 134 and the user input device 130 may be integrated as a touchscreen display.

The display system 134 may be any device capable of communicating visual information to a user. For example, a display system 134 may include a liquid crystal display, a light emitting diode display, and/or any suitable display or displays. The display system 134 can be operable to display information from the signal processor 132 and/or archive 138, such as 2D image slices of the ultrasound volume extracted by the volume navigation processor 140, measurements provided by the measurement processor 160, labels provided by the label processor 170, and/or any suitable information.

The signal processor 132 may be one or more central processing units, microprocessors, microcontrollers, and/or the like. The signal processor 132 may be an integrated component, or may be distributed across various locations, for example. The signal processor 132 comprises a volume navigation processor 140, a segmentation processor 150, a measurement processor 160, and a label processor 170, as described above with reference to FIG. 1, and may be capable of receiving input information from a user input device 130 and/or archive 138, generating an output displayable by a display system 134, and manipulating the output in response to input information from a user input device 130, among other things. The signal processor 132, volume navigation processor 140, segmentation processor 150, measurement processor 160, and/or label processor 170 may be capable of executing any of the method(s) and/or set(s) of instructions discussed herein in accordance with the various embodiments, for example.

The archive 138 may be one or more computer-readable memories integrated with the medical workstation 300 and/or communicatively coupled (e.g., over a network) to the medical workstation 300, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores ultrasound volumes, 2D image slices, instructions for extracting and separately presenting 2D image slices from an ultrasound volume, instructions for automatically segmenting selected follicle(s) in the ultrasound volume, instructions for measuring selected and segmented follicles in 2D image slice(s) and/or the ultrasound volume, and/ or instructions for labeling selected, segmented, and measured follicles in 2D image slice(s), among other things.

The user input device 130 may include any device(s) capable of communicating information from a user and/or at the direction of the user to the signal processor 132 of the medical workstation 300, for example. As discussed above with respect to FIG. 1, the user input device 130 may include a touch panel, button(s), a mousing device, keyboard, rotary encoder, trackball, touch pad, camera, voice recognition, and/or any other device capable of receiving a user directive.

Still referring to FIG. 2, the training system 200 may comprise a training engine 210 and a training database 220. The training engine 210 may comprise suitable logic, circuitry, interfaces and/or code that may be operable to train the neurons of the deep neural network(s) (e.g., artificial intelligence model(s)) inferenced (i.e., deployed) by the segmentation processor 150. For example, the artificial intelligence model inferenced by the segmentation processor 150 may be trained to automatically identify follicle(s) or other anatomical objections of interest depicted in an ultrasound volume using database(s) 220 of classified ultrasound volumes of follicles(s) or anatomical objects of interest.

In various embodiments, the databases 220 of training images may be a Picture Archiving and Communication System (PACS), or any suitable data storage medium. In certain embodiments, the training engine 210 and/or training image databases 220 may be remote system(s) communicatively coupled via a wired or wireless connection to the medical workstation 300 as shown in FIG. 2. Additionally and/or alternatively, components or all of the training system 200 may be integrated with the medical workstation 300 in various forms.

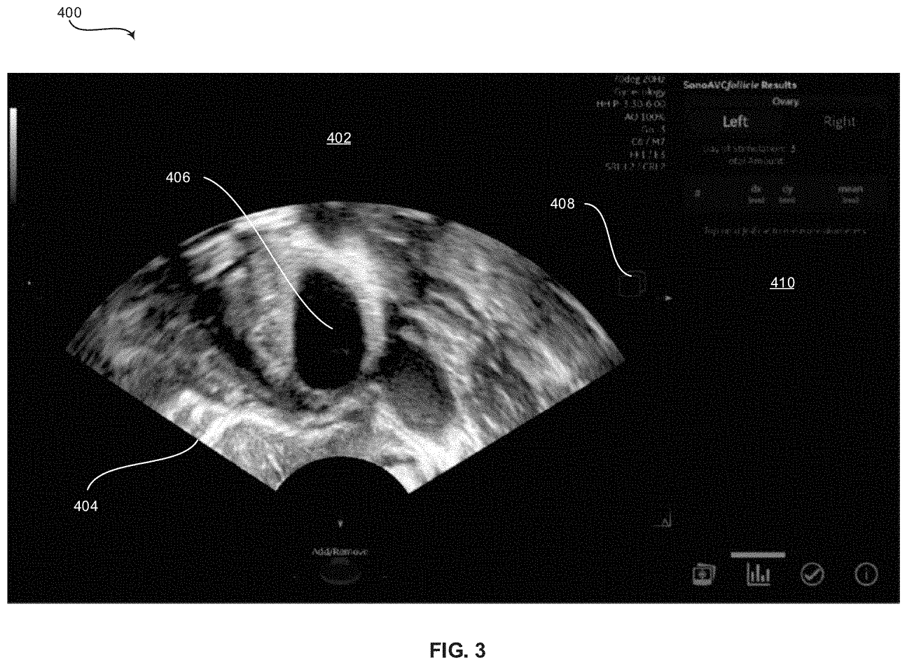

FIG. 3 is an exemplary display 400 of a two-dimensional (2D) image slice 404 of an ultrasound volume depicting a first follicle 406 of an ovary, in accordance with various embodiments. Referring to FIG. 3, the display 400 may comprise an image display portion 402 and a measurement display portion 410. The image display portion 402 may comprise a 2D image slice 404 and a navigation control 408. The 2D image slice 404 may be a 2D image slice extracted from an ultrasound volume of an anatomical object of interest, such as an ovary, by the volume navigation processor 140. The 2D image slice 404 may depict one or more follicles 406 present in the ultrasound volume of the ovary. An ultrasound operator may navigate through a plurality of parallel 2D image slices 404 of the ultrasound volume to identify follicles 406 to measure via a user input device 130. For example, an ultrasound operator may interact with the navigation control 408 via the user input device 130 to advance or rewind to a 2D image slice 404 illustrating a follicle 406 at a largest size. The user input device 130 may be a touchscreen or mousing device operated by an ultrasound operator to navigate between 2D image slices of the ultrasound volume. Additionally and/or alternatively, an ultrasound operator may operate a user input device 130 independent of the navigation control 408 presented at the display 400, such as using a mouse wheel, rotary encoder, and/or any suitable user input device 130 to advance and/or rewind, one at a time, through the plurality of 2D image slices 404 of the ultrasound volume. The measurement display portion 410 may include a list of measurements performed by the measurement processor 160 of the ultrasound system 100 or medical workstation 300.

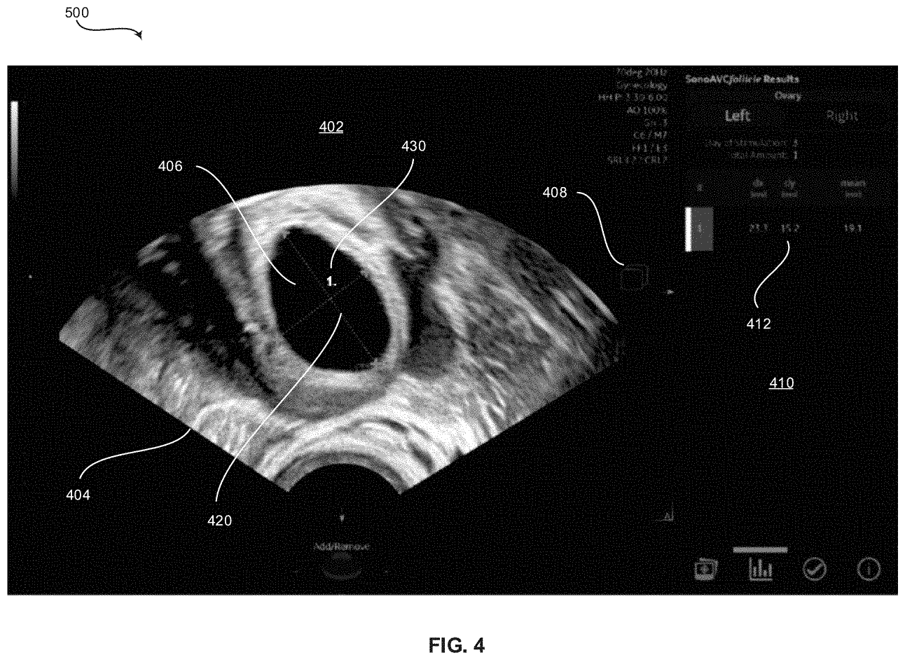

FIG. 4 is an exemplary display 500 of a two-dimensional (2D) image slice 404 of an ultrasound volume depicting a first follicle 406 of an ovary being measured 410, 412, 420 and labeled 430, in accordance with various embodiments. Referring to FIG. 4, the display 500 may comprise an image display portion 402 and a measurement display portion 410. The image display portion 402 may comprise a 2D image slice 404 and a navigation control 408. Once an ultrasound operator navigates to a 2D image slice 404 depicting a follicle 406 at a largest size via a user input device 130 and/or navigation control 408 as described with respect to FIG. 3, the ultrasound operator may select the follicle 406 to be segmented, measured, and labeled in the 2D image slice 404. The user input device 130 may be a touchscreen, mousing device, and/or any suitable user input device 130 operable to select the follicle 406. The selected follicle 406 may be segmented by the segmentation processor 150, measured 410, 412, 420 by the measurement processor 160, and labeled 430 by the label processor 170. For example, as shown in FIG. 4, the measurement processor 160 may be configured to automatically overlay measurement calipers 420 on the selected and segmented follicle 406 in the 2D image slice presented 500 at the display system 134 to obtain perpendicular height and width diameter measurements. In various embodiments, the measurement processor 160 may further determine a mean of the height and width diameter measurements and/or any suitable measurement (e.g., a volume measurement). The measurements 412 may be presented in a measurement display portion 410 of the display 500. The label processor 170 may be configured to superimpose a label 430 at or near the selected follicle 406 to show that the follicle 406 has been measured.

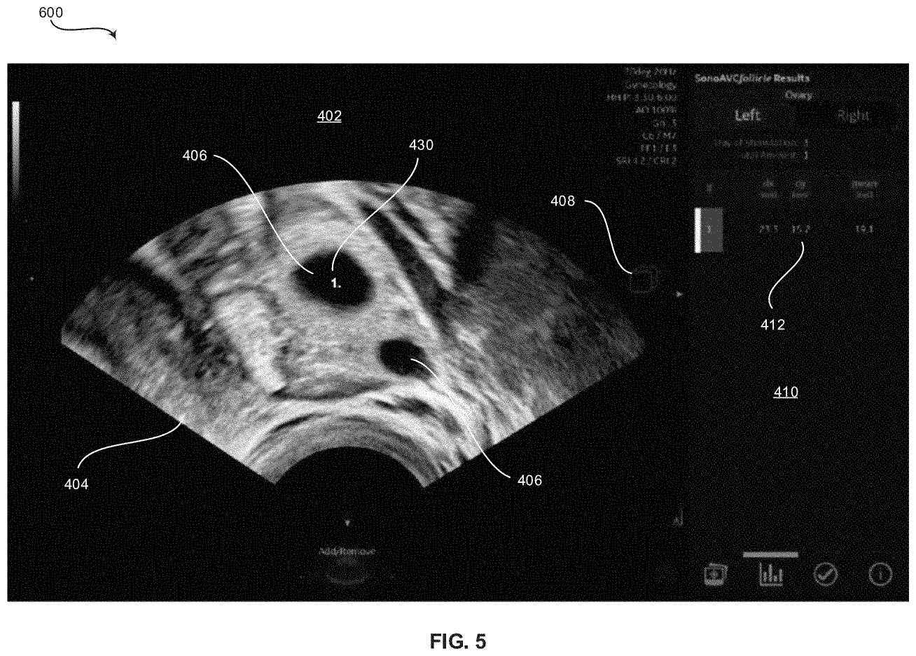

FIG. 5 is an exemplary display 600 of a two-dimensional (2D) image slice 404 of an ultrasound volume depicting a first measured 410, 412, 420 and labeled 430 follicle 406 and a second follicle 406 of an ovary, in accordance with various embodiments. Referring to FIG. 5, the display 600 may comprise an image display portion 402 and a measurement display portion 410. The image display portion 402 may comprise a 2D image slice 404 and a navigation control 408. The measurement display portion 410 may include a list of measurement 412 of labeled 430 follicles 406 that have been measured. Continuing from FIG. 4, an ultrasound operator may continue navigating through the plurality of 2D image slices 404 via the navigation control 408 and/or user input device 130 to identify additional unmeasured follicles 406 at a largest size. Follicles 406 that were previously measured continue to be labeled 430 to avoid double-counting of the follicles 406.

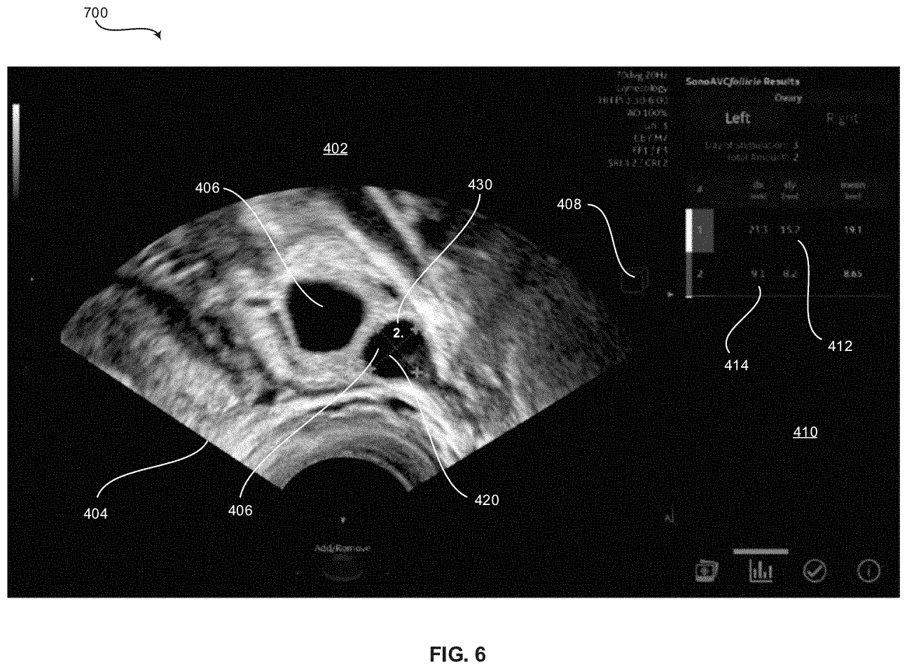

FIG. 6 is an exemplary display 700 of a two-dimensional (2D) image slice 404 of an ultrasound volume depicting a second measured 410, 414, 420 and labeled follicle 406 and a third follicle 406 of an ovary, in accordance with various embodiments. Referring to FIG. 6, the display 700 may comprise an image display portion 402 and a measurement display portion 410. The image display portion 402 may comprise a 2D image slice 404 and a navigation control 408. Continuing from FIG. 5, the ultrasound operator may select a second follicle 406 to be segmented, measured, and labeled in the 2D image slice 404 once an ultrasound operator navigates to a 2D image slice 404 depicting the second follicle 406 at a largest size via a user input device 130 and/or navigation control 408,. The selected second follicle 406 may be segmented, measured 410, 412, 420, and labeled 430. For example, as shown in FIG. 6, the measurement processor 160 may be configured to automatically superimpose measurement calipers 420 on the selected and segmented second follicle 406 in the 2D image slice presented 700 at the display system 134 to obtain perpendicular height and width diameter measurements. In various embodiments, the measurement processor 160 may further determine a mean of the height and width diameter measurements and/or any suitable measurement (e.g., a volume measurement). The measurements 414 may be presented in a measurement display portion 410 of the display 700 along with any other measurements 412 performed during the examination. The label processor 170 may be configured to superimpose a label 430 at or near the selected second follicle 406 to show that the second follicle 406 has been measured. In various embodiments, the measurements 412, 414 presented at the measurement display portion 410 may be differentiated based on a same numerical, textual, symbol, and/or any suitable label overlaid on the 2D image slice 406 at the image display portion 402.

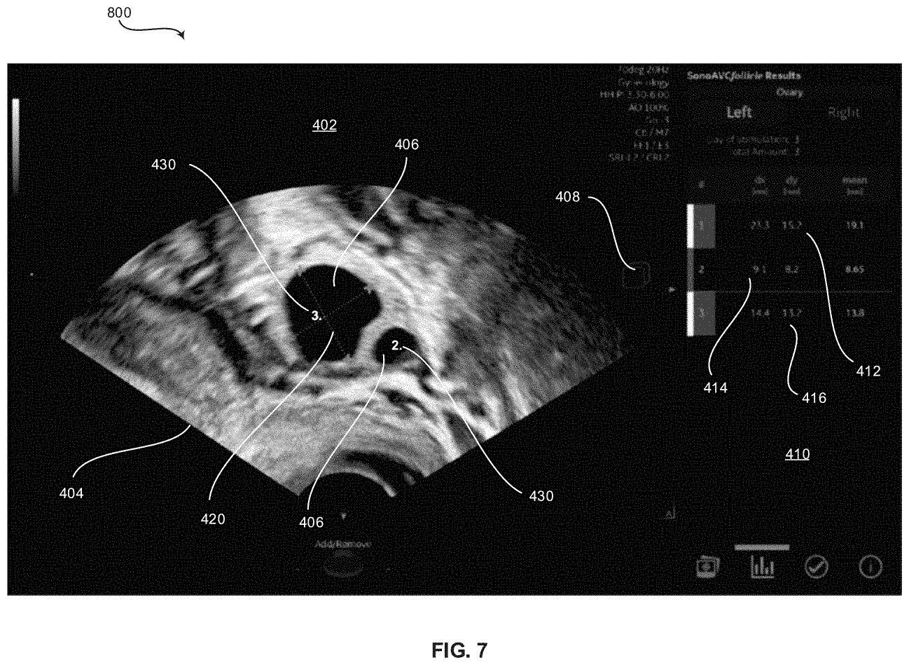

FIG. 7 is an exemplary display 800 of a two-dimensional (2D) image slice 404 of an ultrasound volume depicting second and third measured 410, 414, 416, 420 and labeled 430 follicles 406 of an ovary, in accordance with various embodiments. Referring to FIG. 7, the display 800 may comprise an image display portion 402 and a measurement display portion 410. The image display portion 402 may comprise a 2D image slice 404 and a navigation control 408. Continuing from FIG. 6, the ultrasound operator may select a third follicle 406 to be segmented, measured, and labeled in the 2D image slice 404 once an ultrasound operator navigates to a 2D image slice 404 depicting the third follicle 406 at a largest size via a user input device 130 and/or navigation control 408. The selected third follicle 406 may be segmented, measured 410, 412, 420, and labeled 430. For example, as shown in FIG. 7, the measurement processor 160 may be configured to automatically superimpose measurement calipers 420 on the selected and segmented third follicle 406 in the 2D image slice 404 presented 800 at the display system 134 to obtain perpendicular height and width diameter measurements. In various embodiments, the measurement processor 160 may further determine a mean of the height and width diameter measurements and/or any suitable measurement (e.g., a volume measurement). The measurements 416 may be presented in a measurement display portion 410 of the display 800 along with any other measurements 412, 414 performed during the examination. The label processor 170 may be configured to superimpose a label 430 at or near the selected third follicle 406 to show that the third follicle 406 has been measured. In various embodiments, the measurements 412, 414, 416 presented at the measurement display portion 410 may be differentiated based on a same numerical, textual, symbol, and/or any suitable label overlaid on the 2D image slice 406 at the image display portion 402.

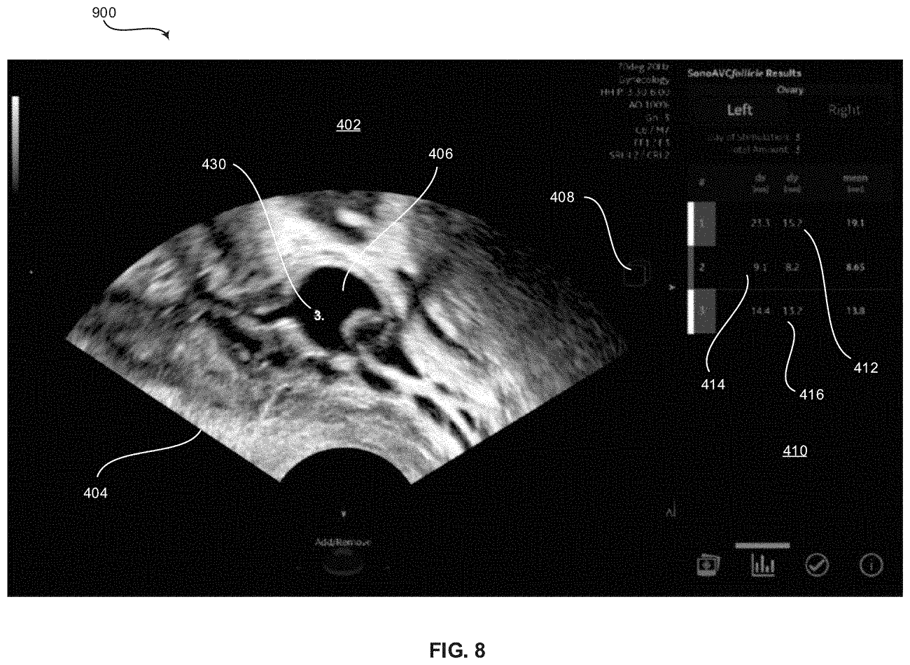

FIG. 8 is an exemplary display 900 of a two-dimensional (2D) image slice 404 of an ultrasound volume depicting a third measured 410, 416, 420 and labeled 430 follicle 406 of an ovary, in accordance with various embodiments. Referring to FIG. 8, the display 900 may comprise an image display portion 402 and a measurement display portion 410. The image display portion 402 may comprise a 2D image slice 404 and a navigation control 408. The measurement display portion 410 may include a list of measurement 412, 414, 416 of labeled 430 follicles 406 that have been measured. Continuing from FIG. 7, an ultrasound operator may continue navigating through the plurality of 2D image slices 404 via the navigation control 408 and/or user input device 130 to identify additional unmeasured follicles 406 at a largest size until the examination is complete (e.g., no additional follicles 406 to measure). Follicles 406 that were previously measured continue to be labeled 430 to avoid double-counting of the follicles 406.

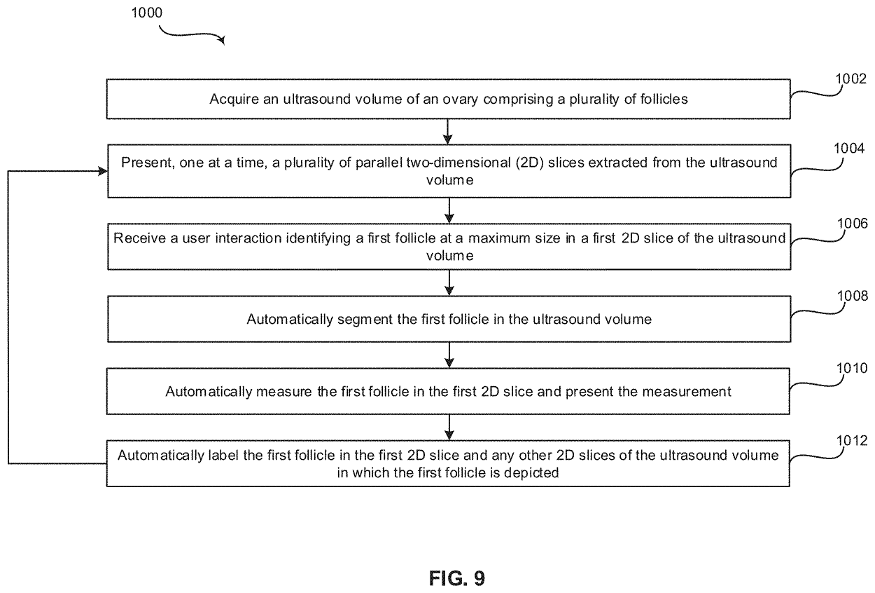

FIG. 9 is a flow chart 1000 illustrating exemplary steps 1002-1012 that may be utilized for automatically measuring 410, 412, 414, 416, 420 and labeling 430 follicles 406 depicted in two-dimensional (2D) image slices 404 of an ultrasound volume, in accordance with various embodiments. Referring to FIG. 9, there is shown a flow chart 1000 comprising exemplary steps 1002 through 1012. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 1002, an ultrasound probe 104 of an ultrasound system 100 acquires an ultrasound volume of an ovary comprising a plurality of follicles 406. For example, the ultrasound probe 104 may be a mechanically wobbling ultrasound probe comprising a one dimensional (1D) array of piezoelectric elements mounted on a transducer assembly movable in a single plane. The ultrasound probe 104 may be operated in a volume acquisition mode, where the transducer assembly of the mechanically wobbling ultrasound probe 104 is automatically moved to acquire a plurality of parallel 2D image slices 404, and where the plurality of 2D image slices 404 form the ultrasound volume, such as an ultrasound volume of an ovary having a plurality of follicles 406. In an exemplary embodiment, the ultrasound probe 104 may be a transvaginal ultrasound probe. In various embodiments, a medical workstation 300 may receive and/or retrieve the ultrasound volume acquired by the ultrasound system 100.

At step 1004, a signal processor 132 of the ultrasound system 100 or medical workstation 300 may cause a display system 134 to present a plurality of 2D image slices 404 extracted from an ultrasound volume, one at a time. For example, a volume navigation processor 140 of the signal processor 132 may be configured to extract and separately present 2D image slices 404 from the ultrasound volume at the display system 134 in response to user selections via the user input device 130 to navigate forward and/or backward through the ultrasound volume. As an example, an ultrasound operator may scroll through separately presented 2D image slices 404 to locate follicles 406 at a largest size via a user input device 130 and/or navigation control 408. As another example, the volume navigation processor 140 may be configured to generate a cine loop of extracted 2D image slices 404 for playback at the display system 134. The ultrasound operator may provide pause, play, rewind, and/or fast forward instructions via the user input device 130 to the volume navigation processor 140 to control playback of the cine loop to identify follicles 406 at a largest size.

At step 1006, the signal processor 132 of the ultrasound system 100 or medical workstation 300 may receive a user interaction identifying a first follicle 406 at a maximum size in a first 2D image slice 404 of the ultrasound volume. For example, a segmentation processor 150 of the signal processor 132 may be configured to receive a user selection of a follicle 406 depicted in a 2D image slice 404 via the user input device 130. The user input device 130 may be a touchscreen, mousing device, and/or any suitable user input device 130 operable to select the follicle 406.

At step 1008, the signal processor 132 of the ultrasound system 100 or medical workstation 300 may automatically segment the first follicle 406 in the ultrasound volume. For example, the segmentation processor 150 may be configured to segment the selected follicle 406 in all 2D image slices 404 in which the selected follicle 406 is depicted within the ultrasound volume. In this regard, the segmentation processor 150 may include, for example, artificial intelligence image analysis algorithms, one or more deep neural networks (e.g., a convolutional neural network such as u-net) and/or may utilize any suitable form of artificial intelligence image analysis techniques or machine learning processing functionality configured to provide segmentation of selected follicles 406. The segmentation processor 150 may be configured to provide a measurement processor 160 and a label processor 170 of the signal processor 132 with the image segmentation information.

At step 1010, the signal processor 132 of the ultrasound system 100 or medical workstation 300 may automatically measure the first follicle 406 in the first 2D image slice 404 of the ultrasound volume and present the measurement 410, 412, 414, 416, 420 at the display system 134. For example, the measurement processor 160 of the signal processor 132 may be configured to receive the segmentation information from the segmentation processor 150 identifying the location and boundaries of the selected first follicle 406 in the 2D image slice 402. The measurement processor 160 may be configured to automatically place measurement calipers 420 on the selected and segmented first follicle 406 in the 2D image slice 404 presented at the display system 134 to obtain perpendicular height and width diameter measurements. In various embodiments, the measurement processor 160 may further determine a mean of the height and width diameter measurements. As another example, the measurement processor 160 may be configured to receive the segmentation information from the segmentation processor 150 identifying the location and boundaries of the selected first follicle 404 in the ultrasound volume. The measurement processor 160 may be configured to determine a volume measurement of selected and segmented first follicle 406 in the ultrasound volume. The measurement processor 160 may be configured to cause the display system 134 to present the 2D follicle diameter measurements 410, 412, 414, 416, 420, volume measurement, and/or any suitable measurement.

At step 1012, the signal processor 132 of the ultrasound system 100 or medical workstation 300 may automatically label 430 the first follicle 406 in the first 2D image slice 404 and any other 2D image slices 404 of the ultrasound volume in which the first follicle 406 is depicted. For example, the label processor 170 may be configured to receive the segmentation information from the segmentation processor 150 identifying the location and boundaries of the selected first follicle 406 in the ultrasound volume. The label processor 170 may be configured to present a different label 430 for each segmented follicle 406 in each of the 2D image slices 404 of the ultrasound volume presented at the display system 134 that includes the segmented follicle 406. The label 430 may be textual, numerical, symbols, and/or any suitable label to distinguish between different selected, segmented, and measured follicles 406. The label processor 170 may be configured to superimpose the label(s) 430 on or near the corresponding follicle(s) 406 depicted in the 2D image slice 404 presented at the display system 134. The process 1000 may return to step 1004 to continue navigating (step 1004) the 2D image slices 404 of the ultrasound volume until all desired follicles 406 are selected (step 1006), segmented (step 1008), measured 410, 412, 414, 416, 420 (step 1010), and labeled 430 (step 1012), at which point the process 1000 ends.

Aspects of the present disclosure provide a method 1000 and system 100, 300 for automatically measuring 410, 412, 414, 416, 420 and labeling 430 follicles 406 depicted in two-dimensional (2D) image slices 404 of an ultrasound volume. In accordance with various embodiments, the method 1000 may comprise receiving 1002, by at least one processor 132, 140 of a system 100, 300, an ultrasound volume of an ovary comprising a plurality of follicles 406. The method 1000 may comprise causing 1004, by the at least one processor 132, 140, a display system 134 to present, one at a time, a plurality of parallel two-dimensional (2D) image slices 404 extracted from the ultrasound volume. The method 1000 may comprise receiving 1006, by the at least one processor 132, 150, a user indication identifying a first follicle 406 at a maximum size in a first 2D image slice 404 of the ultrasound volume presented at the display system 134. The method 1000 may comprise automatically segmenting 1008, by the at least one processor 132, 150, the first follicle 406 in the ultrasound volume. The method 1000 may comprise automatically measuring 1010, by the at least one processor 132, 160, the first follicle 406 in the first 2D image slice 404 to generate a measurement 410, 412, 414, 416, 420 presented at the display system 134. The method 1000 may comprise automatically providing 1012 a label 430, by the at least one processor 132, 170, superimposed on or near the first follicle 406 in the first 2D image slice 404 presented at the display system 134 and in any other of the plurality of parallel 2D image slices 404 depicting the first follicle 406 that is subsequently presented at the display system 134.

In an exemplary embodiment, the measurement 410, 412, 414, 416, 420 comprises 2D follicle diameter measurements 410, 412, 414, 416, 420 in the first 2D image slice 404. In a representative embodiment, the measurement 410, 412, 414, 416, 420 comprises a volume measurement in the ultrasound volume. In various embodiments, the system 100, 300 comprises an ultrasound system 100. In certain embodiments, the method 1000 comprising acquiring 1002, by a transvaginal mechanically wobbling volume ultrasound probe 104, the ultrasound volume. In an exemplary embodiment, the system 100, 300 comprises a medical workstation 300. In a representative embodiment, the plurality of parallel 2D image slices 404 extracted from the ultrasound volume are presented 1004 as a cine loop at the display system 134.

Various embodiments provide a system 100, 300 for automatically measuring 410, 412, 414, 416, 420 and labeling 430 follicles 406 depicted in two-dimensional (2D) image slices 404 of an ultrasound volume. The system 100, 300 may comprise at least one processor 132, 140, 150, 160, 170 and a display system 134. The at least one processor 132, 140 may be configured to receive an ultrasound volume of an ovary comprising a plurality of follicles 406. The at least one processor 132, 140 may be configured to cause a display system 134 to present, one at a time, a plurality of parallel two-dimensional (2D) image slices 404 extracted from the ultrasound volume. The at least one processor 132, 150 may be configured to receive a user indication identifying a first follicle 406 at a maximum size in a first 2D image slice 404 of the ultrasound volume presented at the display system 134. The at least one processor 132, 150 may be configured to automatically segment the first follicle 406 in the ultrasound volume. The at least one processor 132, 160 may be configured to automatically measure the first follicle 406 in the first 2D image slice 404 to generate a measurement 410, 412, 414, 416, 420 presented at the display system 134. The at least one processor 132, 170 may be configured to automatically provide a label 430 superimposed on or near the first follicle 406 in the first 2D image slice 404 presented at the display system 134 and in any other of the plurality of parallel 2D image slices 404 depicting the first follicle 406 that is subsequently presented at the display system 134. The display system 134 may be configured to present the plurality of parallel 2D image slices 404 extracted from the ultrasound volume, the measurement 410, 412, 414, 416, 420, and the label 430. The plurality of parallel 2D images slices 404 comprises the first 2D image slice 404.

In a representative embodiment, the measurement 410, 412, 414, 416, 420 comprises 2D follicle diameter measurements 410, 412, 414, 416, 420 in the first 2D image slice 404. In various embodiments, the measurement 410, 412, 414, 416, 420 comprises a volume measurement in the ultrasound volume. In certain embodiments, the system 100, 300 comprises an ultrasound system 100. In an exemplary embodiment, the system 100 comprises a transvaginal mechanically wobbling volume ultrasound probe 104 configured to acquire the ultrasound volume. In a representative embodiment, the system 100, 300 comprises a medical workstation 300. In various embodiments, the display system 134 is configured to present the plurality of parallel 2D image slices 404 extracted from the ultrasound volume as a cine loop.

Certain embodiments provide a non-transitory computer readable medium having stored thereon, a computer program having at least one code section. The at least one code section is executable by a machine for causing a system 100, 300 to perform steps 1000. The steps 1000 may comprise receiving 1002 an ultrasound volume of an ovary comprising a plurality of follicles 404. The steps 1000 may comprise causing 1004 a display system 134 to present, one at a time, a plurality of parallel two-dimensional (2D) image slices 404 extracted from the ultrasound volume. The steps 1000 may comprise receiving 1006 a user indication identifying a first follicle 406 at a maximum size in a first 2D image slice 404 of the ultrasound volume presented at the display system 134. The steps 1000 may comprise automatically segmenting 1008 the first follicle 406 in the ultrasound volume. The steps 1000 may comprise automatically measuring 1010 the first follicle 406 in the first 2D image slice 404 to generate a measurement 410, 412, 414, 416, 420 presented at the display system 134. The steps 1000 may comprise automatically providing 1012 a label 430 superimposed on or near the first follicle 406 in the first 2D image slice 404 presented at the display system 134 and in any other of the plurality of parallel 2D image slices 404 depicting the first follicle 406 that is subsequently presented at the display system 134.

In various embodiments, the measurement 410, 412, 414, 416, 420 comprises 2D follicle diameter measurements 410, 412, 414, 416, 420 in the first 2D image slice 404. In certain embodiments, the measurement 410, 412, 414, 416, 420 comprises a volume measurement in the ultrasound volume. In an exemplary embodiment, the system 100, 300 comprises an ultrasound system 100. In a representative embodiment, the system 100, 300 comprises a medical workstation 300. In various embodiments, the plurality of parallel 2D image slices 404 extracted from the ultrasound volume are presented 1004 as a cine loop at the display system 134.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y) }. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for automatically measuring and labeling follicles depicted in two-dimensional (2D) image slices of an ultrasound volume.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
receiving, by at least one processor of a system, an ultrasound volume of an ovary comprising a plurality of follicles;
causing, by the at least one processor, a display system to present, one at a time, a plurality of parallel two-dimensional (2D) image slices extracted from the ultrasound volume;
receiving, by the at least one processor, a user indication identifying a first follicle at a maximum size in a first 2D image slice of the ultrasound volume presented at the display system;
automatically segmenting, by the at least one processor, the first follicle in the ultrasound volume;
automatically measuring, by the at least one processor, the first follicle in the first 2D image slice to generate a measurement presented at the display system; and
automatically providing a label, by the at least one processor, superimposed on or near the first follicle in the first 2D image slice presented at the display system and in any other of the plurality of parallel 2D image slices depicting the first follicle that is subsequently presented at the display system.

2. The method of claim 1, wherein the measurement comprises 2D follicle diameter measurements in the first 2D image slice.

3. The method of claim 1, wherein the measurement comprises a volume measurement in the ultrasound volume.

4. The method of claim 1, wherein the system comprises an ultrasound system.

5. The method of claim 4, comprising acquiring, by a transvaginal mechanically wobbling volume ultrasound probe, the ultrasound volume.

6. The method of claim 1, wherein the system comprises a medical workstation.

7. The method of claim 1, wherein the plurality of parallel 2D image slices extracted from the ultrasound volume are presented as a cine loop at the display system.

8. A system comprising:
at least one processor configured to:
receive an ultrasound volume of an ovary comprising a plurality of follicles;
cause a display system to present, one at a time, a plurality of parallel two-dimensional (2D) image slices extracted from the ultrasound volume;
receive a user indication identifying a first follicle at a maximum size in a first 2D image slice of the ultrasound volume presented at the display system;
automatically segment the first follicle in the ultrasound volume;
automatically measure the first follicle in the first 2D image slice to generate a measurement presented at the display system; and
automatically provide a label superimposed on or near the first follicle in the first 2D image slice presented at the display system and in any other of the plurality of parallel 2D image slices depicting the first follicle that is subsequently presented at the display system; and
a display system configured to present:
the plurality of parallel 2D image slices extracted from the ultrasound volume, wherein the plurality of parallel 2D images slices comprises the first 2D image slice;
the measurement; and
the label.

9. The system of claim 8, wherein the measurement comprises 2D follicle diameter measurements in the first 2D image slice.

10. The system of claim 8, wherein the measurement comprises a volume measurement in the ultrasound volume.

11. The system of claim 8, wherein the system comprises an ultrasound system.

12. The system of claim 11, comprising a transvaginal mechanically wobbling volume ultrasound probe configured to acquire the ultrasound volume.

13. The system of claim 8, wherein the system comprises a medical workstation.

14. The system of claim 8, wherein the display system is configured to present the plurality of parallel 2D image slices extracted from the ultrasound volume as a cine loop.

15. A non-transitory computer readable medium having stored thereon, a computer program having at least one code section, the at least one code section being executable by a machine for causing a system to perform steps comprising:
receiving an ultrasound volume of an ovary comprising a plurality of follicles;
causing a display system to present, one at a time, a plurality of parallel two-dimensional (2D) image slices extracted from the ultrasound volume;
receiving a user indication identifying a first follicle at a maximum size in a first 2D image slice of the ultrasound volume presented at the display system;
automatically segmenting the first follicle in the ultrasound volume;
automatically measuring the first follicle in the first 2D image slice to generate a measurement presented at the display system; and
automatically providing a label superimposed on or near the first follicle in the first 2D image slice presented at the display system and in any other of the plurality of parallel 2D image slices depicting the first follicle that is subsequently presented at the display system.

16. The non-transitory computer readable medium of claim 15, wherein the measurement comprises 2D follicle diameter measurements in the first 2D image slice.

17. The non-transitory computer readable medium of claim 15, wherein the measurement comprises a volume measurement in the ultrasound volume.

18. The non-transitory computer readable medium of claim 15, wherein the system comprises an ultrasound system.

19. The non-transitory computer readable medium of claim 15, wherein the system comprises a medical workstation.

20. The non-transitory computer readable medium of claim 15, wherein the plurality of parallel 2D image slices extracted from the ultrasound volume are presented as a cine loop at the display system.
